(19) United States
(12) Patent Application Publication
(10) Pub. No.: US 2010/0184847 A1
(43) Pub. Date: Jul. 22, 2010

(54) COMPOSITIONS FOR SKIN PROTECTION AND IMPROVEMENT OF SKIN DISEASES CONTAINING THE DIBENZO-P-DIOXINE DERIVATIVES

(75) Inventors: Hyeon-Cheol Shin, Daejeon Metropolitan City (KR); Hye-Jeong Hwang, Daejeon Metropolitan City (KR)

Correspondence Address: HAMRE, SCHUMANN, MUELLER & LARSON, P.C., P.O. BOX 2902, MINNEAPOLIS, MN, 55402-0902, US

(73) Assignee: LIVECHEM, INC., Jeju-city, Jeju (KR)

(21) Appl. No.: 12/670,062

(22) PCT Filed: Jul. 30, 2008
(86) PCT No.: PCT/KR08/04450

§ 371 Date: Jan. 21, 2010

(30) Foreign Application Priority Data
Jul. 31, 2007 (KR) 10-2007-0076852

(51) Int. Cl. A61K 31/335; C07D 319/14; A61K 8/49; A61Q 19/00; A61Q 19/08; A61P 17/00

(52) U.S. Cl. 514/452; 549/359; 549/358

(57) ABSTRACT

The present invention relates to compositions for skin protection and improvement that contain dibenzo-p-dioxine derivatives as effective components. Since the compositions for skin protection and improvement that contain the dibenzo-p-dioxine derivative according to the present invention have excellent functions such as moisturizing and/or wrinkle prevention which are useful in prevention and improvement of various skin diseases, they can be extensively used as cosmetic ingredients or pharmaceutical agents for prevention and improvement of skin diseases.

### TECHNICAL FIELD

The present invention provide to compositions which contain dibenzo-p-dioxine derivatives for skin protection and improvement. More particularly, the present invention relates to the compositions comprising dibenzo-p-dioxine derivatives having skin-moisturizing effect and/or wrinkle prevention effect

### BACKGROUND ART

The skin acts as a barrier to protect the internal organs and tissues of the body from physical, chemical, or bacteriological attacks. In addition, it helps to keep the body temperature under control, and prevents loss of water from the inside of the body. The skin has two main structural layers: the epidermis and the dermis. The epidermis is the surface layer of the skin and the dermis is deeper layer providing the structural support of the skin.

The epidermis consists of layers of cells. The bottom layers adjacent to the dermis are the basal cells which reproduced. The top cell layers of skin are called stratum corneum (SC) and cells in SC are longer viable and continuously replaced by new cells. SC receives water from the inside of body and some from the environment. Natural moisturizing factors are generated in SC so that SC acts as a water-retaining barrier. The water content of SC is normally about 30% of its weight. Therefore, the loss of water through SC is responsible for the dry skin. Moisturizing creams and emollients usually help to prevent dryness of skin and to restore normal hydration. That is, skin hydration appears to be the one of most important characteristics of healthy skin, and the major objective of skin pharmacology and cosmetic development is to restore normal hydration.

Among the several reasons, the biosynthesis of melanin by UV exposure is a primary cause of pigmentation in skin. In melanin biosynthetic pathway, DOPA quinine is produced by tyrosinase and then it affords to the black pigment, melanin, through the spontaneous and sequential enzyme reactions. Reduction of melanin levels by inhibiting some of melanin biosynthetic steps may be a general strategy for preventing skin pigmentation. Vitamin C, kojic acid, arbutin, hydroquinone, and several plant extracts such as Moriradicis cortex, have been recently used for these purposes. However, there are limitations in uses of these compounds due to their adverse effects to the skin.

Skin aging is a complex process that involves intrinsic and exogenous causes. Intrinsic skin aging is associated with chronic damage by irreversible degeneration of the tissue, whereas exogenous aging is caused by UV exposure. UV irradiation is the major environmental cause of skin damage and induces skin alternation such as edema. In addition, chronic UV irradiation results in the formation of inflammatory cytokines, degradation of collagen fiber, hyperproliferation of ketatocyte and dysregualation of melanocyte homeostasis, causing wrinkling, roughness, dryness, laxity, and pigmentation. The UV exposure produces pro-inflammatory cytokines such as interleukins (IL-1, IL-6, IL-8, and IL-10), tumor necrosis factor-α (TNF-α). Pro-inflammatory cytokines induced by UV stimulate upregualtion of gene expressions, such as matrix metalloprotease-1 (MMP-1) causing degradation of collagen fibers, basic fibroblast growth factor (bFGF) which promotes hyperproliferation of melanocytes and keratocytes, and MAPK (mitogen activated protein kinase). Therefore, the effective inhibition of these pro-inflammatory cytokines should be useful for the prevention of skin from UV-induced inflammation.

Elastase is the typical enzyme associated with skin aging and capable of degrading elastin, an elastic fibrous protein in animal tissues. Elastase activity can be stimulated by intrinsic aging or external UV exposure. Increase in elastase level result in over-productions of elastin which are responsible for the degeneration of collagen fibers network, wrinkle formation, and decrease of skin elasticity. Especially, skin elasticity remarkably decreases after 40 years of age due to overexpression of elastase. Elevations of elastase activity by chronological aging result in degradation and aggregation of elastic fiber, and reduction of collagen synthesis. Physiologically, expressions of elastase have been promoted by chronological aging. Therefore, the effective inhibition of the elastase activity should be useful for preventing formation of wrinkle.

The acne vulgaris is an inflammatory disease in sebaceous glands in the skin, which often occurs in pubertal young individuals under hormone influence. Acne is characterized by excess sebum production and enlargement of the sebaceous glands which are activated by the androgen, proliferation of keratocytes in sebaceous glands, comedogenesis associated with hypercornification of the follicular wall epidermis, and inflammation by microbial species, Propionibacterium acnes (P. acnes). Various types of retinoic acids (RAs) have been known as acne therapy to inhibit the sebum production and enlargement of sebaceous gland. In addition, it has been reported that antibiotics are effective in the treatment of acne due to inhibition of the proliferation of P. acnes. However, the use of these RAs and antibiotics in acne therapy has been limited in acceptance due to their adverse effects, such as skin inflammation, irritation and the induction of bacterial resistance. Therefore, developing effective and safe anti-acne agents to reduce sebum and prevent inflammation is highly desirable.

### DISCLOSURE

### Technical Problem

Therefore, an object of the present invention is to provide nontoxic compositions for prevention and improvement of skin diseases. In detail, an object of the present invention is to provide compositions having skin-moisturizing and/or wrinkle prevention effects.

Another object of the present invention is to provide cosmetics that contain the compositions for protection of skin and improvement of skin disease.

### Technical Solution

According to an aspect of the present invention, provided are compositions for preventing skin diseases and improving skin disease symptoms that comprise dibenzo-p-dioxine(dibenzo-p-dioxine) derivatives which possess wrinkle-preventing and/or skin-moisturizing functions.

According to another aspect of the present invention, provided are cosmetics containing the compositions that have excellent functions on skin protection and/or improvement of skin diseases.

According to still another aspect of the present invention, provided are pharmaceutical agents containing the compositions that protect skin and/or treat skin diseases.

### ADVANTAGEOUS EFFECTS

As described above, since the compositions comprising the dibenzo-p-dioxine derivatives of the present invention are non-toxic extract from seaweeds, and show excellent effects in prevention and treatment of various skin diseases, they can be valuably used as pharmaceutical agents for protecting skin and improving skin diseases and/or cosmetic ingredients.

### DESCRIPTION OF DRAWINGS

FIG. 1 is a graph that illustrates the anti-inflammation and anti-allergic effect of the compositions represented by the change in flare area;

FIG. 2 is a graph that illustrates the anti-inflammation and anti-allergic effect of the compositions represented by the change in weal volume;

FIG. 3 is a graph that shows the inhibitory effects of the compositions on pro-inflammatory gene expression induced by UV irradiation;

FIG. 4 is a graph that illustrates the dose-response curve of revertant colonies by the compositions in differential bacterial strains (without metabolism activation system); and

FIG. 5 is a graph that illustrates the dose-response curve of revertant colonies by the composition in differential bacterial strains (with metabolism activation system)

### BEST MODE

Hereinafter, the present invention will be described in detail.

The present inventors have found that compositions comprising dibenzo-p-dioxine derivatives are effective in prevention and treating various skin diseases, thereby accomplished the present invention.

The dibenzo-p-dioxine derivatives that are comprised in the composition of the present invention were first found in edible kelps. In the present invention it was discovered that the compositions of the present invention comprising the dibenzo-p-dioxine derivatives possess: skin-whitening function by suppressing melanine biosynthesis; wrinkle-preventing function by suppressing elastase activity; anti-inflammatory and anti-allergic functions by suppressing histamine activity; moisture retention function; acne improvement function by suppressing the generation of sebum; prevention and improvement of various skin diseases by protection from skin aging and inflammation generated by reactive oxygen and ultraviolet rays. In addition, in the present invention, it was found that toxicity does not occur according to the use of the skin agent over a long period of time.

As a representative compound of the dibenzo-p-dioxine derivatives that are useful in the present invention, there are following compounds shown in Formula 1 through Formula 10.

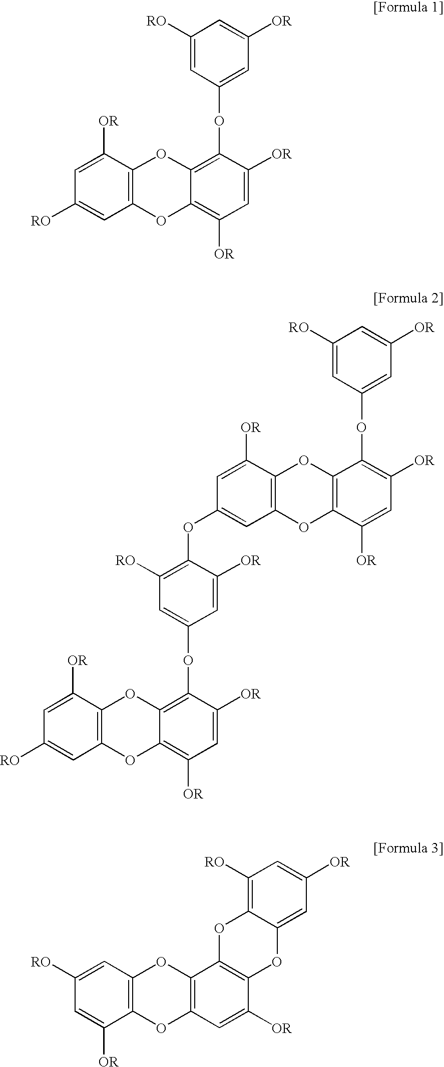

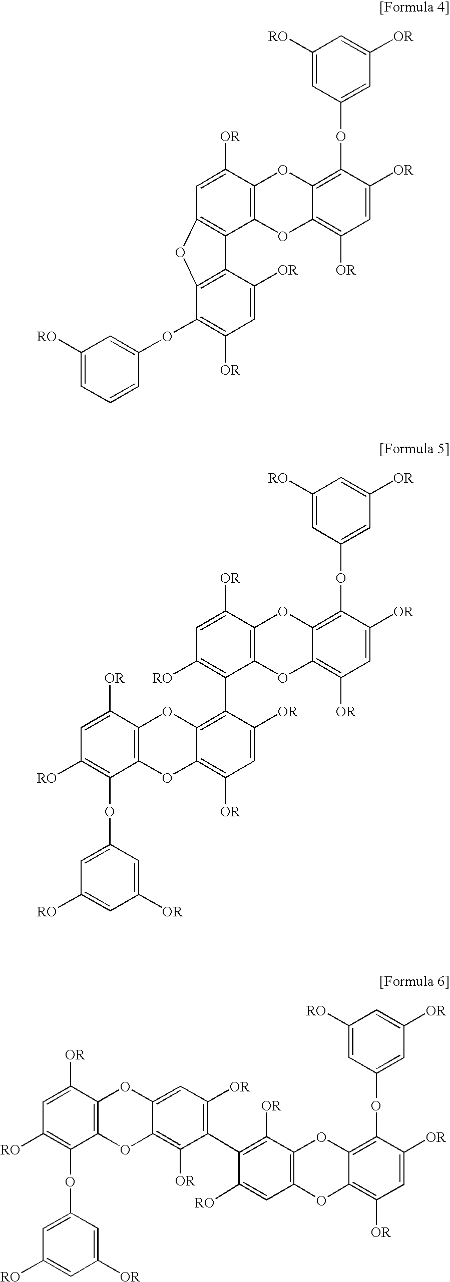

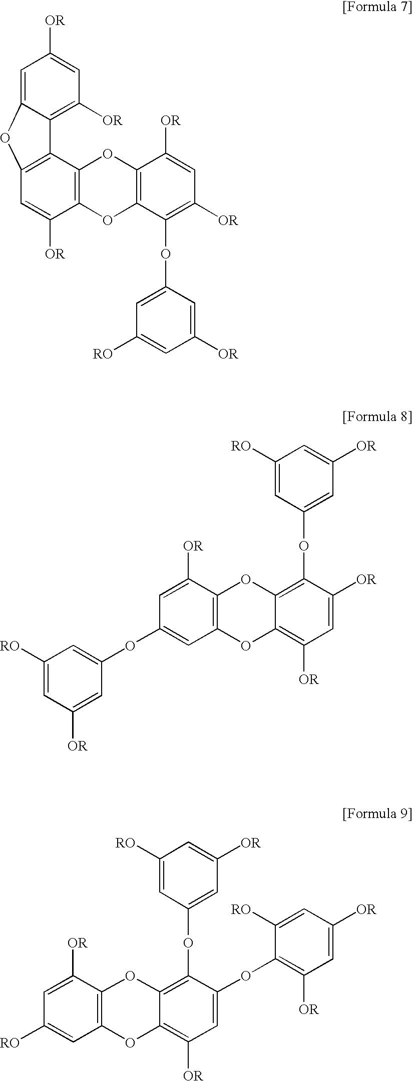

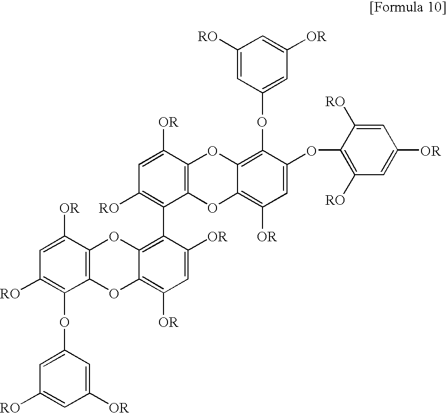

wherein each R is H, alkyl, alkenyl, phenyl, phenyl alkyl, alkanoyl, hydroxyphenyl, dihydroxyphenyl, or acyl. Preferably, each R is H.

The compositions according to the present invention may comprise at least one dibenzo-p-dioxine derivative. For example, the dibenzo-p-dioxine derivative may comprise 8-90% by weight of at least one dibenzo-p-dioxine derivative that is selected from the group consisting of the dibenzo-p-dioxine derivative of Formula 2 and the dibenzo-p-dioxine derivative of Formula 4, and 10-92% by weight of at least one dibenzo-p-dioxine derivative that is selected from the group consisting of the dibenzo-p-dioxine derivative of Formula 1, the dibenzo-p-dioxine derivative of Formula 3, the dibenzo-p-dioxine derivative of Formula 5, the dibenzo-p-dioxine derivative of Formula 6, the dibenzo-p-dioxine derivative of Formula 7, the dibenzo-p-dioxine derivative of Formula 8, the dibenzo-p-dioxine derivative of Formula 9, and the dibenzo-p-dioxine derivative of Formula 10.

In addition, the dibenzo-p-dioxine derivative may comprise 0.1-6% by weight of the dibenzo-p-dioxine derivative of Formula 1, 5-60% by weight of the dibenzo-p-dioxine derivative of Formula 2, 1-30% by weight of the dibenzo-p-dioxine derivative of Formula 3, 0.5-20% by weight of the dibenzo-p-dioxine derivative of Formula 4, 0.1-10% by weight of the dibenzo-p-dioxine derivative of Formula 5, 0.5-15% by weight of the dibenzo-p-dioxine derivative of Formula 6, 0.1-5% by weight of the dibenzo-p-dioxine derivative of Formula 7, 0.1-5% by weight of the dibenzo-p-dioxine derivative of Formula 8, 0.1-10% by weight of the dibenzo-p-dioxine derivative of Formula 9, and 0.1-12% by weight of the dibenzo-p-dioxine derivative of Formula 10 while at least two thereof may be mixed with each other.

The daily dosage of the composition may be in the range of 1-100 mg/Kg.

The dibenzo-p-dioxine derivative may be extracted from the kelp, and specifically from Eisenia bicyclis, Eisenia arborea, Eisenia desmarestioides, Eisenia galapagensis, Eisenia masonii, Ecklonia kurome, Ecklonia cava, Ecklonia stolonifera, Ecklonia maxima, Ecklonia radiata, Ecklonia bicyclis, Ecklonia biruncinate, Ecklonia buccinalis, Ecklonia caepaestipes, Ecklonia exasperta, Ecklonia fastigiata, Ecklonia brevipes, Ecklonia arborea, Ecklonia latifolia, Ecklonia muratii, Ecklonia radicosa, Ecklonia richardiana or Ecklonia wrightii. Preferably, the dibenzo-p-dioxine derivative is extracted from Eisenia bicyclis, Ecklonia cava, Ecklonia kurome or Ecklonia stolonifera.

The content of this dibenzo-p-dioxine derivative is not particularly limited, but it may be comprised in the range of 0.00001-100% by weight in the composition according to the present invention.

As the cosmetic for skin protection and improvement that comprises the dibenzo-p-dioxine derivative according to the present invention, there are a base cosmetic products (lotion, cream, essence, cleansing foam, cleansing water, pack, body oil), a color cosmetic products (foundation, lipstick, mascara, makeup base), a hair cosmetic material (shampoo, rinse, hair conditioner, hair gel) and the like.

In addition, the composition according to the present invention may be produced in a form that is capable of being allowed as a pharmaceutical product.

In the cosmetic that comprises the composition according to the present invention, the composition may be comprised in the range of 0.00001-50% by weight.

In the medical product that comprises the composition according to the present invention, the composition may be comprised in the range of 0.001-100% by weight.

### MODE FOR INVENTION

Hereinafter, the present invention will be described with reference to the following Examples, but are not to be construed to limit the present invention.

### Example 1

### Production of Extracts and Separation of Single Compounds from Seaweeds

After the Ecklonia cava and the Eisenia bicyclis were washed with the distilled water to remove the impurity, they were dried in the darkened room and then cut into small pieces. Mixture of 500 g of seaweeds (Ecklonia cava 350 g, and Eisenia bicyclis 150 g) and 20 times of 10% alcohol was refluxed for 2 hours. This process was repeated two times. Extracts were filtered and concentrated using rotary evaporator under reduced pressure. Extracts were diluted with 20 times of distilled water and added with ethyl acetate. The ethyl acetate fraction was separated from water. This process was repeated three times. Combined ethyl acetate fractions were concentrated under reduced pressure and then loaded into the silica gel column (15 times of concentrate). Crude extract containing dibenzo-p-dioxine derivatives was obtained using ethyl acetate/acetone (volume ratio 9/1) as an eluent.

The crude extract was filtered using the 0.2 μm membrane filter and loaded into the high speed liquid chromatography (HPLC). The single compounds (Formula 1 to 10) were separated using HPLC (column: HP ODS Hypersil; eluent: 15%-70% of aquous methanol, linear gradient; flow rate: 1.0 ml/min).

### Example 2

### Production of the Compositions 1 to 18

The compositions 1 to 18 were produced from the single compounds (Formulas 1 to 10). The chemical composition of the compositions 1 to 18 is described in Table 1.

TABLE 1
Chemical composition of the compositions 1 to 18

| Sample | Composition of the sample |
|---|---|
| composition 1 | I (R═H), 100% |
| composition 2 | II (R═H), 100% |
| composition 3 | III (R═H), 100% |
| composition 4 | IV (R═H), 100% |
| composition 5 | V (R═H), 100% |
| composition 6 | VI (R═H), 100% |
| composition 7 | VII (R═H), 100% |
| composition 8 | VIII (R═H), 100% |
| composition 9 | IX (R═H), 100% |
| composition 10 | X (R═H), 100% |
| composition 11 | II (R═H), 60% + III (R═H), 25% + IV (R═H), 15% |
| composition 12 | IV (R═H), 70% + V (R═H), 8% + VI (R═H), 22% |
| composition 13 | IV (R═H), 10% + X (R═H), 80% + VII (R═H), 10% |
| composition 14 | I (R═H), 3% + II (R═H), 60% + III (R═H), |
| | 10% + IV (R═H), 12% + V (R═H), |
| | 5% + VI (R═H), 10% |
| composition 15 | II (R═H), 60% + IV (R═H), 20% + VI (R═H), |
| | 15% + VII (R═H), 5% |
| composition 16 | IV (R = acetyl, H (3:7)), 100% |
| composition 17 | II (R = oleoyl, H (1:9)), 100% |
| composition 18 | VI (R = methyl, H (2:8)), 100% |

### Example 3

### Inhibition of Melanin Synthesis

To demonstrate the whitening effect of the compositions in the present invention, inhibitory effects of the compositions 1 to 18 (test group) on melanin synthesis were investigated. Catechin, rasveratrol, isoflavone, kojic acid, ascorbic acid, and Moriradicis cortex extracts were used as positive controls.

The B-16 cells (mouse melanoma, ATCC CRL 6323) were maintained in the DMEM medium supplemented 4.5 g/l of glucose, 10% serum, and 1% antibiotic at 37° C. for 24 hours. After the cell was incubated with 0.05% trypsin containing 0.02% EDTA, the cell was plated and incubated for 48 hours. The cells were treated with 50 μg/ml of compositions 1 to 18, or positive controls and incubated at 37° C. for 3 days. The cells were then added with 1 ml of lysis buffer (phosphate buffer solution, 0.02% EDTA, 0.05% trypsin) and centrifuged for 5 min. After the cells were treated with 5% trichloro acetate (TCA), the formed melanin was separated and dissolved in 1N NaOH. The absorbance was measured at 475 nm using spectrophotometer. Concentration of melanin was determined by the standard curve of synthetic melanin (SIGMA CO. USA). The cell treated with the same amount of solvent was used as a negative control. The inhibitory effects of each composition on melanin synthesis were determined by following Equation. The results are described in Table 2.

{1−(M/M₀)}×100 [Equation]

M: amount of melanin of test group or positive controls

M0: amount of melanin of negative control

TABLE 2
Inhibitory effects of the compositions on melanin synthesis

| | | Inhibitory |
|---|---|---|
| | | effect on |
| | | melanin |
| | | synthesis |
| Sample | Composition of the sample | (%) |
| composition 1 | I (R═H), 100% | 84.3 |
| composition 2 | II (R═H), 100% | 82.0 |
| composition 3 | III (R═H), 100% | 88.0 |
| composition 4 | IV (R═H), 100% | 82.3 |
| composition 5 | V (R═H), 100% | 83.5 |
| composition 6 | VI (R═H), 100% | 89.8 |
| composition 7 | VII (R═H), 100% | 80.5 |
| composition 8 | VIII (R═H), 100% | 87.6 |
| composition 9 | IX (R═H), 100% | 84.3 |
| composition 10 | X (R═H), 100% | 92.1 |
| composition 11 | II (R═H), 60% + III (R═H), | 94.2 |
| | 25% + IV (R═H), 15% | |
| composition 12 | IV (R═H), 70% + V (R═H), 8% + VI | 92.8 |
| | (R═H), 22% | |
| composition 13 | IV (R═H), 10% + X (R═H), 80% + VII | 92.8 |
| | (R═H), 10% | |
| composition 14 | I (R═H), 3% + II (R═H), 60% + III | 94.4 |
| | (R═H), 10% + IV (R═H), 12% + V | |
| | (R═H), 5% + VI (R═H), 10% | |
| composition 15 | II (R═H), 60% + IV (R═H), 20% + VI | 94.4 |
| | 15% + VII (R═H), 5% | |
| composition 16 | IV (R = acetyl, H (3:7)), 100% | 92.4 |
| composition 17 | II (R = oleoyl, H (1:9)), 100% | 95.7 |
| composition 18 | VI (R = methyl, H (2:8)), 100% | 94.0 |
| catechin | | 50.4 |
| rasveratrole | | 40.9 |
| isoflavone | | 64.3 |
| kojic acid | | 60.3 |
| ascorbic acid | | 65.3 |
| moriradicis cortex | | 30.6 |
| extract | | |

As shown in Table 2, melanin levels of test group were significantly reduced by 83-94% (positive controls; 30-65%). It is confirmed that the compositions 1 to 18 exhibited remarkable inhibitory effect on melanin synthesis when compared with positive controls. Therefore, our data show that the compositions comprised the dibenzo-p-dioxine derivatives have an excellent whitening effect which associated with inhibition of melanin synthesis.

### Example 4

### Inhibition of Elastase

To demonstrate the wrinkle prevention effect of the compositions in the present invention, inhibitory effect of the compositions 1 to 18 (test group) on elastase were investigated. Catechin, rasveratrol, isoflavone, lactokine were used for positive controls.

50 μg/ml of test group or positive controls were treated with 10 nM of elastase at 25° C. for 10 min, and then absorbance was measured at 410 nm. The cell treated with the same amount of solvent was used as a negative control. The inhibitory effects of each sample on elastase were determined by following Equation. The results are described in Table 3.

{1−(E/E0)}×100 [Equation]

E: The absorbance of test group or positive control

E0: The absorbance of negative control

TABLE 3
Inhibition of elastase

| | | Inhibition |
|---|---|---|
| | | of |
| | | Elastase |
| Sample | Composition of the sample | (%) |
| composition 1 | I (R═H), 100% | 87.4 |
| composition 2 | II (R═H), 100% | 86.4 |
| composition 3 | III (R═H), 100% | 83.2 |
| composition 4 | IV (R═H), 100% | 85.4 |
| composition 5 | V (R═H), 100% | 90.4 |
| composition 6 | VI (R═H), 100% | 89.9 |
| composition 7 | VII (R═H), 100% | 88.7 |
| composition 8 | VIII (R═H), 100% | 87.7 |
| composition 9 | IX (R═H), 100% | 82.7 |
| composition 10 | X (R═H), 100% | 88.1 |
| composition 11 | II (R═H), 60% + III (R═H), 25% + IV | 90.4 |
| | (R═H), 15% | |
| composition 12 | IV (R═H), 70% + V (R═H), 8% + VI | 93.5 |
| | (R═H), 22% | |
| composition 13 | IV (R═H), 10% + X (R═H), 80% + VII | 91.9 |
| | (R═H), 10% | |
| composition 14 | I (R═H), 3% + II (R═H), 60% + III | 95.0 |
| | (R═H), 10% + IV (R═H), 12% + V | |
| | (R═H), 5% + VI (R═H), 10% | |
| composition 15 | II (R═H), 60% + IV (R═H), 20% + VI | 88.0 |
| | (R═H), 15% + VII (R═H), 5% | |
| composition 16 | IV (R = acetyl, H (3:7)), 100% | 94.9 |
| composition 17 | II (R = oleoyl, H (1:9)), 100% | 94.9 |
| composition 18 | VI (R = methyl, H (2:8)), 100% | 92.1 |
| catechin | | 50.4 |
| rasveratrole | | 48.3 |
| isoflavone | | 38.3 |
| ractocaine | | 37.8 |

As shown in Table 3, inhibitions of elastase by the test group were significantly increased by 82-93% (positive controls; 30 to 65%). It is confirmed that the composition 1 to 18 showed remarkable inhibitory effect on elastase when compared with positive controls. Therefore, our data demonstrate that the compositions comprised the dibenzo-p-dioxine derivatives have an excellent wrinkle prevention effect which associated with inhibition of elastase.

### Example 5

### Anti-Inflammation and the Anti-Allergic Activity: Inhibition on Histamin Synthesis

In order to assess the anti-inflammation and anti-allergic activities of the compositions in the present invention, the inhibitory effects of the compositions on histamine synthesis, which closely related with inflammation including allergy, were investigated.

The inhibitory effects of the compositions on histamine synthesis were assessed using the rat basophilic leukemia cell (RBL-2H3) according to the Kawasaki's method. After the cell (1×105 cells/well) was cultured in RPMI 1640 medium supplemented with 2% FBS (fetal bovine serum) and rat anti-DNP (dinitrophenol) IgE at 37° C. for 120 min, the cell was washed with the HPEPS buffer to remove the residual IgE. The cells was incubated in either absence (negative control) or presence (test group) of the compositions 1 to 18 (50 μg/ml) at 37° C. for 10 min. The cell was then treated with DNP-BSA (Dinitrophenol—conjugated Bovine serum albumin), as a histamine release antigen, for 1 hour at 37° C. After the reaction was stopped by adding the HEPES buffer solution, the supernatant was collected and treated with 20 μl of the perchloric acid and centrifuged. The histamine release in the supernatant was measured using the HPLC. The % inhibitions on histamine release of test group were calculated by following Equation compared with negative control. The results are showed in Table 4.

{1−(H/H₀)}×100 [Equation]

H: histamine release of test group

H0: histamine release of negative control

TABLE 4
% Inhibition on histamine release

| | | % |
|---|---|---|
| Sample | Composition of the sample | Inhibition |
| composition 1 | I (R═H), 100% | 60.4 |
| composition 2 | II (R═H), 100% | 70.3 |
| composition 3 | III (R═H), 100% | 58.9 |
| composition 4 | IV (R═H), 100% | 80.3 |
| composition 5 | V (R═H), 100% | 77.4 |
| composition 6 | VI (R═H), 100% | 75.9 |
| composition 7 | VII (R═H), 100% | 70.8 |
| composition 8 | VIII (R═H), 100% | 62.9 |
| composition 9 | IX (R═H), 100% | 72.1 |
| composition 10 | X (R═H), 100% | 83.2 |
| composition 11 | II (R═H), 60% + III (R═H), 25% + IV | 85.9 |
| | (R═H), 15% | |
| composition 12 | IV (R═H), 70% + V (R═H), 8% + VI | 89.8 |
| | (R═H), 22% | |
| composition 13 | IV (R═H), 10% + X (R═H), 80% + VII | 90.3 |
| | (R═H), 10% | |
| composition 14 | I (R═H), 3% + II (R═H), 60% + III | 80.5 |
| | (R═H), 10% + IV (R═H), 12% + V | |
| | (R═H), 5% + VI (R═H), 10% | |
| composition 15 | II (R═H), 60% + IV (R═H), 20% + VI | 88.3 |
| | (R═H), 15% + VII (R═H), 5% | |
| composition 16 | IV (R = acetyl, H (3:7)), 100% | 80.2 |
| composition 17 | II (R = oleoyl, H (1:9)), 100% | 92.1 |
| composition 18 | VI (R = methyl, H (2:8)), 100% | 89.0 |

As shown in Table 4, inhibitions of histamine synthesis by test group were significantly increased compared with negative control. Therefore, our data show that the compositions comprised the dibenzo-p-dioxine derivatives have an excellent anti-inflammatory and anti-allergy activities associated with inhibition of histamine synthesis.

### Example 6

### Anti-Inflammation and Anti-Allergic Activities: Anti-Inflammation Activity Against Histamine-Induced Inflammation

Anti-inflammation efficacy of the compositions against histamine-induced skin inflammation was clinically investigated.

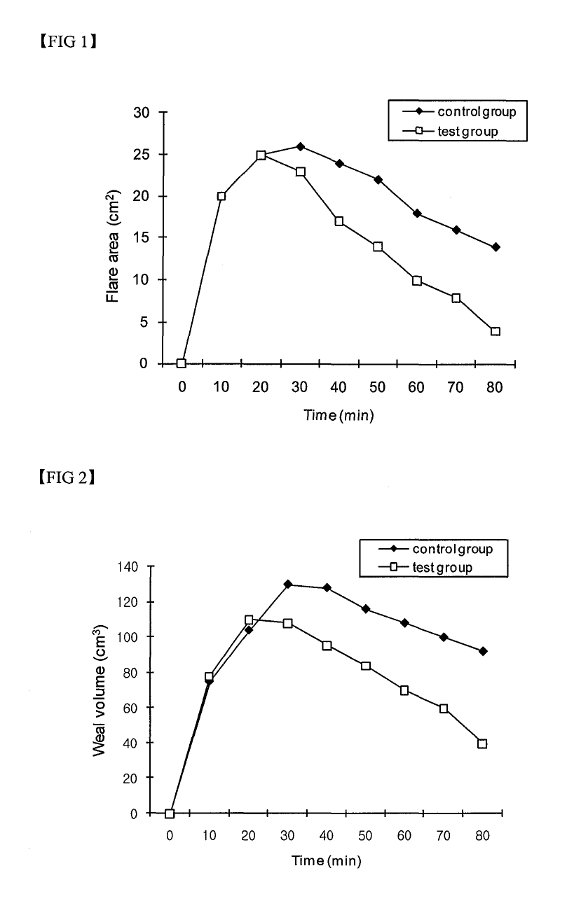

TABLE 5
Test method

| | |
|---|---|
| Subject | 21 participants without the medical history such as the eczema, |
| | psoriasis, atopic dermatitis, etc. (16 female, 5 male, average age 37 years, |
| | range 23-56) |
| Sample | control group: cream not contain the compositions |
| | test group: cream contain 5% of the composition 14 |
| Method | 50 μl of histamine (100 μg/ml) was injected intradermally into the inner |
| | forearm skin of both arms. |
| | After 10 min, the resulting weal and flare were measured at 10 min |
| | intervals for 20 min |
| | After 20 min, 200 μl of the creams (test and control) were topically |
| | applied to cover the flare and weal on the experimental arm. |
| | The testing skin areas were measured at 10 min intervals for 40 min. |
| Assessment | Weal skin thickness (mm) and flare area were calculated by the |
| | following equation, and the results were illustrated in FIG. 1. |
| | Flare area (cm2) = π/4 × (D1 + D2)²/2 |
| | Weal volume (cm3) = π/4 × (d1 + d2)/2 × (Tt − T0)/2 |
| | D1: diameter of flare |
| | D2: second perpendicular diameter of flare |
| | d1; diameter of weal |
| | d2: second perpendicular diameter of weal |
| | Tt: skinfold thickness at time t min |
| | T0: skinfold thickness at time 0 |

As shown in FIGS. 1 and 2, data demonstrate that the composition have excellent anti-inflammatory effect on the histamine-induced skin inflammation.

### Example 7

### Assessment of Skin Hydration Capacity

The skin-moisturizing effects of the compositions were clinically evaluated using human skin.

TABLE 6
Test method

| | |
|---|---|
| Subject | 80 healthy participants (44 female, 36 male; average ages 56 ± 5.6 years) |
| Sample | Control group: cream not contain the compositions |
| | Test group: cream contain 1% of composition 15 |
| Method | All participants were topically applied with creams on one of forearm 2 |
| | times daily (morning and evening) for the 60 days. |
| Assessment | Baseline, after 30 and 60 days |
| time | |
| Assessment | 1. Skin surface hydration index |
| method | Skin surface hydration was assessed with a Corneometer CM 825 |
| | (Courage et Khazaka, Cologne, Germany) to measure dielectrical |
| | constant which is the characterized by each compound. Since the |
| | dielectric constants of carotene and fat are different from each other, |
| | the dielectric constant of the keratin layer can be changed by skin |
| | hydration. Therefore, skin surface hydration can be determined. |
| | 2. TEWL(trans epidermal water loss) |
| | TEWL was determined using an Evaporimeter EP1 (Servomed, |
| | Stockholm, Sweden) following the guidelines from the European |
| | Society of Contact Dermatitis. TEWL indicates the diffusion of water |
| | through the stratum corneum (SC). It is measured by the estimated |
| | vapor-pressure gradient within an open chamber. |

TABLE 7
Test result

| Assessment | | | |
|---|---|---|---|
| method | Baseline | After 30 days | After 60 days |
| Skin surface | | | |
| hydration | | | |
| index (AU) | | | |
| All (n = 80) | 33.98 | 44.17 (30% | 52.91 (53% |
| | | increase) | increase) |
| Male (n = 36) | 33.33 | 45.00 (35% | 54.32 (63% |
| | | increase) | increase) |
| Female (n = 44) | 34.50 | 43.34 (26% | 51.75 (50% |
| | | increase) | increase) |
| TEWL (g/m²h) | | | |
| All (n = 80) | 9.64 | 6.94 (28% decrease) | 5.49 (43% decrease) |
| Male (n = 36) | 9.28 | 6.03 (35% decrease) | 4.82 (48% decrease) |
| Female (n = 44) | 9.93 | 7.68 (22% decrease) | 6.04 (39% decrease) |

As shown in Table 7, data demonstrate that the composition have excellent skin-moisturizing effect through increasing skin hydration capacity and preventing skin dryness.

### Example 8

### Prevention and Treatment of Acne

The prevention and treatment effects of the compositions on the acne were assessed through the inhibition of TG (triacyl glycerol) synthesis and sebocyte proliferation in hamster model.

### Example 8-1

### Inhibition of TG synthesis

The inhibitory effects of the composition 6 and 14 on TG synthesis were assessed as compared with catechin (positive control group). 5 week-old male hamster was divided into three groups (negative control group, test group, positive control group) and housed 10 animals to a cage after the acclimatization for 2 weeks. Animal rooms were maintained at 25±1° C. and 55% of humidity with a 12-hr light/dark cycle. The animals were freely accessed to diet and water throughout the study. The auricles of hamster were topically treated once a day for 14 days with 200 μl of 10% of the composition 6, 14 or catechin. The solvent for topical application was composed of a mixture of ethanol and glycerol (95:5, v/v). The animals in negative control group were treated with the same volume of vehicle. The auricles were separated from the body and the sebum generated on the skin surface was extracted with acetone. The amount of TG in skin surface was calculated from the sebum extracts. Sebocyte were suspended into solution from the auricle tissues using sonicator, and then amount of intercellar TG was determined by automatic thin-layer chromatography using triolate as a standard. The relative TG concentrations of each sample were calculated from comparison with negative control. The results are described in Table 8.

TABLE 8
Reduction of sebum generation

| | Relative TG | Relative TG |
|---|---|---|
| | concentration in | concentration in |
| sample | the skin surface (%) | the skin cell (%) |
| control group | 100 | 100 |
| composition 6 | 66.2 | 54.9 |
| solution | | |
| composition 14 | 35.0 | 26.9 |
| solution | | |
| catechin | 108.2 | 110.4 |

### 8-2

### Suppression of Sebocyte Proliferation

Hamster sebocyte were established from sebaceous glands of auricles of the 5 week-old hamsters according to Sato's method. Sebocytes (2.35×10⁴ cells/plate) were cultured in DMEM/F12 medium supplemented with 2% FBS and 2% human serum for 24 hours. The cells were treated with the composition 6 and 14 in different concentrations one time per 3 days for 12 days. The [³H]thymidine (1 kBq/well) (Amersham Bioscience) was added 3 hours before the final treatment. The amount of [³H]thymidine combined with DNA was measured by using liquid scintillation counter. The cell proliferation activity was determined as compared with negative control. The results are described in Table 9.

TABLE 9
Suppression of sebocyte proliferation

| | sample | cell number | % of |
|---|---|---|---|
| sample | concentration | (×10³ d.p.m/well) | untreated cell |
| control group | | 2.90 ± 0.20 | 100 |
| composition 6 | 10 | 2.26 ± 0.67 | 78.4 |
| solution | 20 | 0.87 ± 0.07 | 30.2 |
| | 40 | 0.30 ± 0.05 | 10.4 |
| composition | 0.1 | 1.57 ± 0.37 | 50.4 |
| 14 | 0.5 | 0.53 ± 0.21 | 18.3 |
| solution | 1 | 0.22 ± 0.04 | 7.7 |

As shown in Table 8, the composition 6 and 14 significantly decrease sebum production not only on skin surface but also in the skin cell when compared with negative control. As shown in Table 9, the composition 6 and 14 significantly suppressed sebocyte proliferation. Therefore, data show that the compositions containing the dibenzo-p-dioxine derivatives have highly anti-acne effect through the inhibition of sebum production and sebocyte proliferation.

### Example 9

### Suppression of Inflammatory Gene Expression Induced by UV Exposure

The anti-inflammation effects of the compositions were investigated by measuring the UVB-induced generation of pro-inflammatory cytokines (IL-1α, IL-1β, IL-6, IL-8, and TNF-α) in the epidermis.

The normal human epidermal keratinocytes (NHEK cell line) were incubated in the keratinocyte-SFM medium (serum-free medium) at 95% humidity in 5% carbon dioxide at 37° C. The cells were placed (3×10⁴ cells/wall) into the 96-well plate and allowed to adhere 24 hours. The cells were treated with the composition 6, 16 or green tea extract in the concentration of 50 μg/ml. DMSO (dimethyl sulfoxide) was used as a solvent and the final concentration of DMSO was 0.1% (v/v). After incubation for 24 hours, the cells were was irradiated with UVB (40 mJ/cm²). The cultured supernatants were collected 24 hour after UVB irradiation. The concentrations of pro-inflammatory cytokines (IL-1β, IL-6, IL-8, and TNF-α) were determined from Human Inflammation Cytometric Bead Assay kit (Becton Deckinson, San Diego, USA). The concentration of IL-1α was measured using the enzyme immunoassay. The results are described in FIG. 2. The negative control indicates the cells which were neither irradiated nor treated with the compositions, and the positive control means the cells which were only irradiated with UVB without treatment of the compositions.

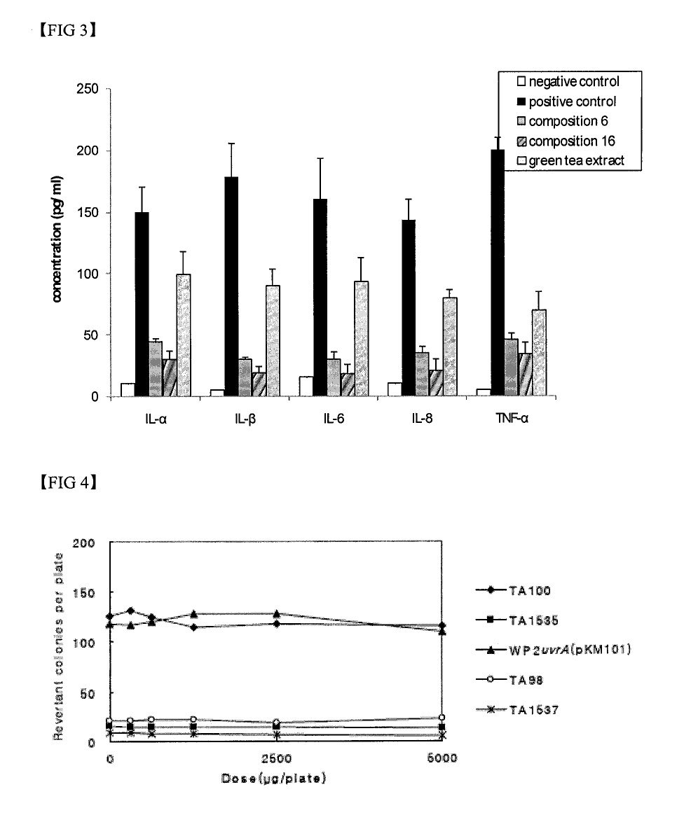

As shown in FIG. 3, the composition 6 and 16 significantly decrease levels of pro-inflammatory cytokines when compared with positive control. Therefore, data show that the compositions containing the dibenzo-p-dioxine derivatives have highly preventive effects against UV-induced skin damages.

### Example 10

### Toxicity Test on Human Skin Cell

The cytotoxicity of the compositions to the normal skin cell was assessed using the NHEK (F) cells (normal human epidermal keratinocytes of neonatal foreskin cell) and the NB1RGB cells (normal human fibroblast cell line from skin). NHEK (F) and NB1RGB cells, which are main components of the epidermis and demis in normal human skin, produce collagen and elastin and provide skin elasticity.

After the NHEK (F) cell and the NB1 GB cell were incubated in RPMI 1640 medium, the compositions were treated in different concentrations and incubated for 1 hour. The mixture of PMS/WST-1 (1-methoxy-5-methylphenazinium methyl sulfate/2-(4-indophenyl)-3-(4-nitrophenyl)-5-(2,4-disulfo phenyl)-2H-tetrazolium monosodium salt) was added to the medium and incubated for 1 hour to generate soluble formosan, and then the absorbance was measured at 415 nm using spectrophotometer. The cells treated with solvent without the compositions were used as a negative control, whereas the cells treated with 1% (w/v) SDS solution were used as a positive control (100% cell apoptosis).

The cytotoxicity (apoptosis, %) was calculated by the following Equation and represented by LD₅₀ (Lethal dose 50, the concentration reaches to 50% of apoptosis, μM) of each composition. The results are described in Table 9.

apoptosis(%)=(A0−A/A0−As)×100 [Equation]

A0=absorbance of the cell that was not treated with the composition

A=absorbance of the cell that was treated with the composition (negative control group)

As=absorbance of the cell that was treated with 1% (w/v) SDS (positive control group)

TABLE 10
Toxicity evaluation in respects to the skin cell

| | | LD₅₀ (μM) | |
|---|---|---|---|
| | | NHEK (F) | NB1RG |
| Sample | Composition of the sample | cell | B cell |
| composition 1 | I (R═H), 100% | 56.4 | 110.4 |
| composition 2 | II (R═H), 100% | 48.9 | 150.3 |
| composition 3 | III (R═H), 100% | 55.0 | 159.8 |
| composition 4 | IV (R═H), 100% | 96.3 | 149.3 |
| composition 5 | V (R═H), 100% | 100.4 | 139.4 |
| composition 6 | VI (R═H), 100% | 105.4 | 97.3 |
| composition 7 | VII (R═H), 100% | 78.9 | 100.4 |
| composition 8 | VIII (R═H), 100% | 93.2 | 104.8 |
| composition 9 | IX (R═H), 100% | 57.2 | 114.3 |
| composition 10 | X (R═H), 100% | 48.2 | 128.5 |
| composition 11 | II (R═H), 60% + III (R═H), 25% + IV (R═H), | 84.7 | 137.4 |
| | 15% | | |
| composition 12 | IV (R═H), 70% + V (R═H), 8% + VI (R═H), | 74.3 | 150.3 |
| | 22% | | |
| composition 13 | IV (R═H), 10% + X (R═H), 80% + VII (R═H), | 58.0 | 138.9 |
| | 10% | | |
| composition 14 | I (R═H), 3% + II (R═H), 60% + III (R═H), 10% + | 90.5 | 128.4 |
| | IV (R═H), 12% + V (R═H), 5% + VI (R═H), | | |
| | 10% | | |
| composition 15 | II (R═H), 60% + IV (R═H), 20% + VI (R═H), | 58.2 | 135.3 |
| | 15% + VII (R═H), 5% | | |
| composition 16 | IV (R = acetyl, H (3:7)), 100% | 60.3 | 125.3 |
| composition 17 | II (R = oleoyl, H (1:9)), 100% | 66.5 | |
| composition 18 | VI (R = methyl, H (2:8)), 100% | 70.4 | |

As shown in Table 10, the compositions show very low toxicity to the skin cell.

### Example 11

### Genetic Toxicity Test (Bacterial Reverse Mutation Test)

By using histidine—demand salmonellae and tryptophan—demand Escherichia coli., the reverse mutagenesis of the composition was evaluated.

Each 100 μL of test solution (negative control, the composition 15 and positive control), either 500 μL of 0.1 mol/L sodium phosphate buffer (pH 7.4) (without metabolic activation system) or 500 μl, of S9 mix (with metabolic activation system), and 100 μL of suspension for cultured strains (1×109 cells/mL) were mixed in a dry heat sterilized glass tube (13 mm×100 mm). This mixture was incubated in a shaking water bath at 37° C. for 20 minutes. Then, it was mixed and stirred with 2 mL of warmed top agar (45° C.). Finally, the content of each tube was poured into a Vogel-Bonner minimum glucose agar plate and the overlaid agars were allowed to solidify. The highest dose in the main test was 5000 μg/plate regardless of metabolic activation system and was sequentially diluted by common ratio of 2 to produce 4 additional lower doses. Concurrent negative and positive control groups were included.

After the incubation, the number of revertant colonies per plate was counted automatically by the colony counter (ProtoCOL, SINBIOSIS, UK). The existence of growth inhibition and deposition for the test substance was examined by the background lawn using a microscope.

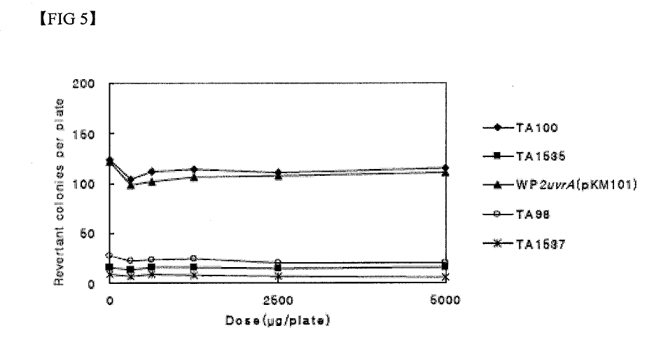

As a result of preliminary dose range-finding test, cytotoxicity and deposition were not observed in all strains regardless of metabolic activation system. As a result, the number of revertant colonies in the test strains was not increased more than two-fold regardless of application of metabolic activation system as compared with that of the negative control group. [see FIGS. 4 and 5] In conclusion, the compositions did not show the mutagenic potential.

Examples of the cosmetic prescriptions containing the composition are as described in the following tables.

TABLE 11
An example of an aqueous cosmetic prescription that contains the
composition

| Component | Content (% by weight) |
|---|---|
| Above composition | 0.1 |
| 1,3-butylene glycol | 6.0 |
| sodium hyaluronate | 2.0 |
| glycerine | 4.0 |
| PEG 4000 | 1.0 |
| polysorbate 20 | 0.5 |
| ethanol | 10.0 |
| antiseptic | predetermined amount |
| benzophenone-9 | 0.05 |
| flavor | predetermined amount |
| purified water | predetermined amount |
| total | 100 |

TABLE 12
A prescription example of a milk lotion that contains the composition

| Component | Content (% by weight) |
|---|---|
| Above composition | 0.1 |
| stearic acid | 0.4 |
| 1,3-butylene glycol | 6.0 |
| cetostearyl alcohol | 1.2 |
| glycerine | 4.0 |
| glyceryl stearate | 1.0 |
| triethanolammine | 0.25 |
| tocopheryl acetate | 3.0 |
| fluid paraffin | 5.0 |
| squalene | 3.0 |
| macadamia nut oil | 2.0 |
| polysorbate 60 | 1.5 |
| sorbitan sesquioleate | 0.6 |
| carboxyvinyl polymer | 0.15 |
| antiseptic | predetermined amount |
| flavor | predetermined amount |
| purified water | residual amount |
| total | 100 |

TABLE 13
A prescription example of nutrition cream that contains the
composition

| Component | Content (% by weight) |
|---|---|
| Above composition | 0.1 |
| petrolatum | 7.0 |
| cetostearyl alcohol | 2.5 |
| glyceryl stearate | 2.0 |
| stearic acid | 1.5 |
| fluid paraffin | 10.0 |
| wax | 2.0 |
| polysorbate 60 | 1.5 |
| sorbitan sesquioleate | 0.8 |
| squalene | 3.0 |
| 1,3-butylene glycol | 6.0 |
| glycerine | 4.0 |
| triethanolammine | 0.5 |
| tocopheryl acetate | 0.1 |
| antiseptic | predetermined amount |
| flavor | predetermined amount |
| purified water | residual amount |
| total | 100 |

TABLE 14
A prescription example of essence that contains the composition

| Component | Content (% by weight) |
|---|---|
| Above composition | 0.1 |
| Glycerine | 10.0 |
| PEG 1500 | 2.0 |
| allantion | 0.1 |
| pantenol | 0.3 |
| EDTA | 0.02 |
| benzophenone-9 | 0.04 |
| hydroxyethyl cellulose | 0.1 |
| sodium hyaluronate | 8.0 |
| carboxyvinyl polymer | 0.2 |
| triethanolammine | 0.18 |
| octyldodeceth-25 | 0.6 |
| ethanol | 6.0 |
| antiseptic, flavor, pigment | small amount |
| purified water | residual amount |
| total | 100 |

TABLE 15
A Prescription example of the massage cream that contains the
composition

| Component | Content (% by weight) |
|---|---|
| Above composition | 0.1 |
| glyceryl stearate | 2.0 |
| cetostearyl alcohol | 2.5 |
| stearic acid | 1.0 |
| polysorbate 60 | 1.5 |
| solbitan stearate | 0.6 |
| isostearyl isostearate | 5.0 |
| squalene | 5.0 |
| mineral oil | 35.0 |
| dimethicone | 1.0 |
| xanthan gum | 0.1 |
| hydroxyethyl cellulose | 0.12 |
| glycerine | 6.0 |
| triethanolammine | 0.5 |
| antiseptic, flavor, pigment | predetermined amount |
| purified water | residual amount |
| total | 100 |

1. Compositions for a wrinkle-preventing and/or moisturizing agents comprising:
one or more dibenzo-p-dioxine derivatives as effective components.

2. The composition as set forth in claim 1, wherein the dibenzo-p-dioxine derivative is one or more that are selected from compounds that are represented by the following Formula 1 to 10: 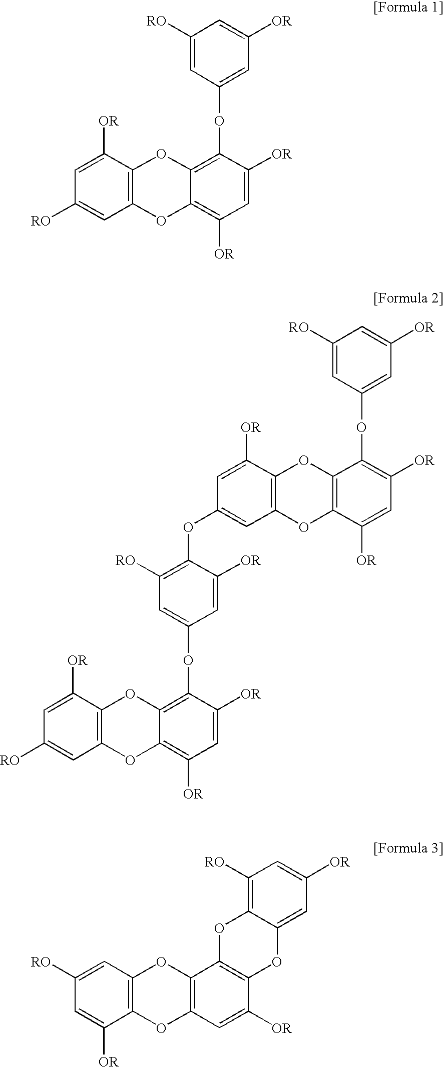 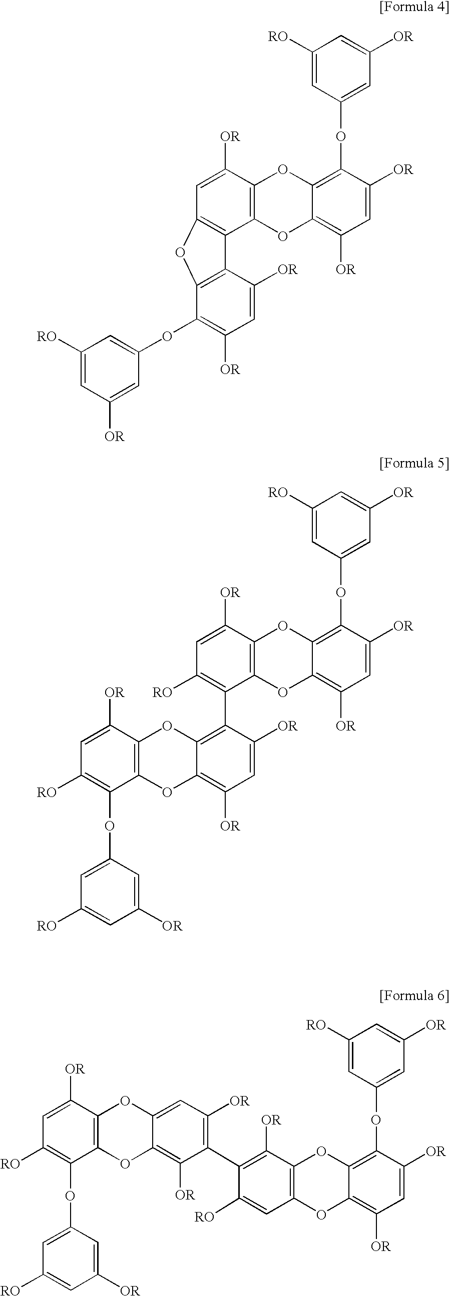 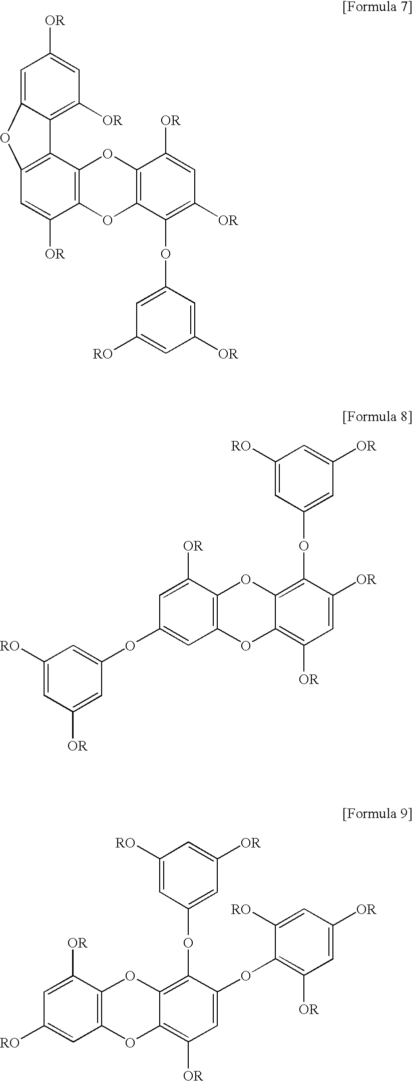 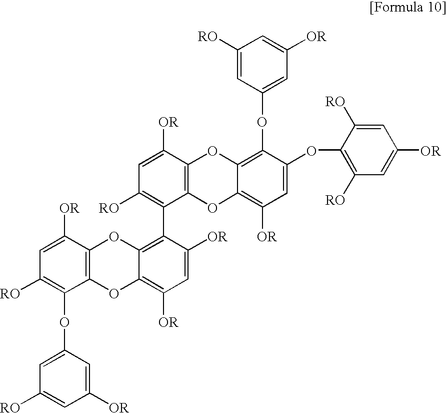
wherein each R is H, alkyl, alkenyl, phenyl, phenyl alkyl, alkanoyl, hydroxyphenyl, dihydroxy phenyl, or acyl.

3. The compositions as set forth in claim 2, wherein R is H.

4. The compositions as set forth in claim 2, wherein the compositions comprise at least two dibenzo-p-dioxine derivatives.

5. The composition as set forth in claim 4, wherein the dibenzo-p-dioxine derivatives comprises 8-90% by weight of at least one dibenzo-p-dioxine derivative that is selected from the group consisting of the dibenzo-p-dioxine derivative of Formula 2 and the dibenzo-p-dioxine derivative of Formula 4, and 10-92% by weight of at least one dibenzo-p-dioxine derivative that is selected from the group consisting of the dibenzo-p-dioxine derivative of Formula 1, the dibenzo-p-dioxine derivative of Formula 3, the dibenzo-p-dioxine derivative of Formula 5, the dibenzo-p-dioxine derivative of Formula 6, the dibenzo-p-dioxine derivative of Formula 7, the dibenzo-p-dioxine derivative of Formula 8, the dibenzo-p-dioxine derivative of Formula 9, and the dibenzo-p-dioxine derivative of Formula 10.

6. The composition as set forth in claim 1, wherein the dibenzo-p-dioxine derivative is extracted from Eisenia bicyclis, Eisenia arborea, Eisenia desmarestioides, Eisenia galapagensis, Eisenia masonii, Ecklonia kurome, Ecklonia cava, Ecklonia stolonifera, Ecklonia maxima, Ecklonia radiata, Ecklonia bicyclis, Ecklonia biruncinate, Ecklonia buccinalis, Ecklonia caepaestipes, Ecklonia exasperta, Ecklonia fastigiata, Ecklonia brevipes, Ecklonia arborea, Ecklonia latifolia, Ecklonia muratii, Ecklonia radicosa, Ecklonia richardiana or Ecklonia wrightii.

7. The compositions as set forth in claim 1, wherein the daily dosage of the compositions are in the range of 1-100 mg/Kg.

8. Cosmetics for skin protection or improvement containing the compositions according to claim 1.

9. Pharmaceutical agents containing the compositions according to claim 1 for prevention and treatment of skin diseases.
